(19) United States
(12) Patent Application Publication
(10) Pub. No.: US 2023/0210774 A1
(43) Pub. Date: Jul. 6, 2023

(54) FOOD GRADE WHITENING AGENTS AND USES THEREOF

(71) Applicant: PHYTO TECH CORP., Rancho Santa Margarita, CA (US)

(72) Inventors: Cuie YAN, San Pedro, CA (US); Jordan Reese FARMER, Foothill Ranch, CA (US); Sang Ryoung KIM, Irvine, CA (US); Daniela Rosa RUIZ, Irvine, CA (US); Steven CHEN, Coto De Caza, CA (US)

(21) Appl. No.: 18/149,804

(22) Filed: Jan. 4, 2023

Related U.S. Application Data
(60) Provisional application No. 63/296,374, filed on Jan. 4, 2022.

(51) Int. Cl. A61K 9/16; A61K 47/14; A61K 9/00

(52) U.S. Cl. CPC A61K 9/16; A61K 47/14; A61K 9/0056; A23L 33/24

(57) ABSTRACT

This disclosure provides MCT-loaded microcapsules for use as whitening agents in food and pharmaceutical products.

### BACKGROUND OF THE INVENTION

### Field of Invention

This disclosure provides a dry powder comprising MCT-loaded microcapsules for use as a whitening agent in food, beverage, and pharmaceutical products.

### Background

One of the most widely used food pigments is titanium dioxide (E171), an odorless powder that enhances the white color or opacity of foods. The most common foods containing titanium dioxide are chewing gum, candies, pastries, chocolates, coffee creamers, and cake decorations. Titanium dioxide is also present in cosmetics, paints, and medicines.

In recent decades, concerns about the risks of titanium dioxide consumption have grown. The Food and Drug Administration (FDA) categorizes titanium dioxide as Generally Recognized as Safe, but other organizations have issued warnings. For example, the European Food Safety Authority (EFSA) has concluded that titanium oxide should not be considered safe as a food additive due to uncertainties about possible inflammation and neurotoxicity. There exists a need for safe alternatives to titanium dioxide for use as whitening agents in consumer products.

### BRIEF SUMMARY OF THE INVENTION

In one aspect, the disclosure provides microcapsules for use as a whitening agent comprising an inner core and a primary shell surrounding the inner core, wherein (i) the inner core comprises food grade medium chain triglyceride oil (MCT); and (ii) the primary shell comprises food grade modified starch.

The microcapsules described above are referred to as “MCT-loaded microcapsules.”

In another aspect, MCT-loaded microcapsules are spray dried and the resulting dry powder is used as a whitening agent in foods and pharmaceutical products. For example, the dry powder comprising MCT-loaded microcapsules can be used to bring a bright and smooth white appearance to pastries or confectionary, or to opacify yogurt or ice cream. The dry powder comprising MCT-loaded microcapsules can also be used as a whitening agent in candy, chewing gum and cake icing. The dry powder comprising MCT-loaded microcapsules can also be used as a whitening agent in beverages, e.g., soy milk. The dry powder comprising MCT-loaded microcapsules can also be used as a whitening agent in pharmaceutical products, e.g., in tablet or capsule preparations.

### DETAILED DESCRIPTION OF THE INVENTION

In one embodiment, the disclosure provides a dry powder for whitening a food, or pharmaceutical product comprising spray-dried particles, wherein:

(i) the spray-dried particles comprise microcapsules; and

(ii) the microcapsules comprise an inner core and a primary shell surrounding the inner core, wherein (a) the inner core comprises food grade MCT; and (b) the primary shell comprises food grade modified starch or gum Arabic.

In another embodiment, the disclosure provides a dry powder for whitening a food, or pharmaceutical product comprising spray-dried particles, wherein:

(i) the spray-dried particles comprise microcapsules.

In another embodiment, the disclosure provides a dry powder described above, wherein the redispersion diameter of the spray-dried particles is about 200 nm to about 300 nm.

In another embodiment, the disclosure provides a dry powder described above having an L* value of at least 95 in the CIELAB color space.

In another embodiment, the disclosure provides a dry powder described above having an a* value less than 0.15 in the CIELAB color space.

In another embodiment, the disclosure provides a dry powder described above having a b* value less than 5.0 in the CIELAB color space.

In another embodiment, the disclosure provides a dry powder described above that is stable. As used herein, the term “stable” may refer to retention of the same or substantially similar size, shape, color, or whitening effect over an extended period of time, e.g, days, weeks, months, or years. In some embodiments, stability can be determined by monitoring on a repeated basis, e.g., monthly, the color and particle size of each whitening powder itself over a certain time period, e.g., 2 years, at a certain temperature, e.g., room temperature, in a sealed bag. The whitening powder can also be tested in various applications, e.g. by tasting them with a focus on creaminess and/or off notes, and recording their whitening effect by color measurement.

In another embodiment, the disclosure provides a beverage comprising a sufficient amount of a dry powder described above to provide whitening to the beverage

In another embodiment, the disclosure provides a food comprising a sufficient amount of a dry powder described above to provide whitening to the food.

In another embodiment, the disclosure provides a pharmaceutical product comprising a sufficient amount of a dry powder described above to provide whitening to the pharmaceutical product.

Unless stated otherwise, the term “microcapsule” as used herein refers to single-core, multicore, or a mixture of single-core and multicore microcapsules.

Particle size can be measured using any typical equipment known in the art, for example, a Zetasizer Ultra, Malvern, Worcestershire, UK.

The microcapsules described in this section are collectively referred to as “Microcapsules of the Disclosure” (each individually referred to as a “Microcapsule of the Disclosure”).

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular

Measuring Color: The three values L*, a*, b* are used to characterize the principal color in the CIELAB model specified by the International Commission on Illumination (Commission Internationale d'Eclairage). It describes all the colors visible to the human eye and was created to serve as a device independent model to be used as a reference. L* denotes the luminosity and extends from 0 (black) to 100 (white). The value a* measures the red and green of the color: the colors tending toward green have a negative value while those tending toward the red have a positive a* value. The b* value measures the blue and the yellow of the color: colors tending toward the yellow have a positive b* value while those tending toward the blue have a negative b* value. The L*, a*, b* values are measured using a spectrum colorimeter.

As used herein, the singular forms “a,” “an,” and “the” include plural references unless the context clearly dictates otherwise. For example, the term “a compound” or “at least one compound” may include a plurality of compounds, including mixtures thereof. The terms “a”, “an,” “the,” “one or more,” and “at least one,” for example, can be used interchangeably herein.

As used herein, the term “about,” means plus or minus 10% of the reported numerical value.

Various embodiments of this disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range, such as from 1 to 6 should be considered to have specifically disclosed subranges such as from 1 to 2, from 1 to 3, from 1 to 4, from 1 to 5, from 2 to 3, from 2 to 4, from 2 to 5, from 2 to 6, from 3 to 4, from 3 to 5, from 3 to 6, etc., as well as individual numbers within that range, for example, 1, 2, 3, 4, 5, and 6. This applies regardless of the breadth of the range.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “comprises,” “comprising,” “includes,” “including,” “having,” and their conjugates are interchangeable and mean “including but not limited to.” It is understood that wherever aspects are described herein with the language “comprising,” otherwise analogous aspects described in terms of “consisting of” and/or “consisting essentially of” are also provided.

The term “consisting of” means “including and limited to.”

The term “consisting essentially of” means the specified material of a composition, or the specified steps of a method, and those additional materials or steps that do not materially affect the basic characteristics of the material or method.

The term “food grade” as used herein refers to material that is safe for human consumption.

A weight percent (wt. %) of a component is based on the total weight of the composition in which the component is included.

### EXAMPLES

### Example 1

Synthesis of Modified Starch-MCT Microcapsules at 20% MCT Loading Level

Step 1. Deionized water (180 g) is heated to 60° C. in a beaker.

Step 2. To this beaker, 72 g of modified starch is added and allowed to fully dissolve with agitation by an overhead mixer. The mixer speed is determined by starch type and total volume, but never exceeds 400 RPM.

Step 3. Once starch is fully dissolved (usually after around 1 hour), 18 g of MCT oil is added to the beaker.

Step 4. This final mixture is added to a jacketed reactor cooled to 4° C. and stirred until core temperature is cooled to around 20° C. to 25° C.

Step 5. The reactor is moved to a polytron homogenizer and emulsified at 20,000 RPM for 10 minutes. The emulsion droplet size is monitored until the average is around 1 μm. At this point, emulsion is ready for spray drying.

Step 6. Characterize resulting spray dry powder by color, final particle size, and re-dispersibility.

Color Measurements of Powders of Example 1 Using the CIELAB Model

TABLE 1

| | | | | Redispersion |
|---|---|---|---|---|
| | Color measurement | | | diameter |
| Sample ID | L* | a* | b* | nm |
| Titanium Dioxide (reference) | 96.06 | −0.26 | 1.54 | 310.5 |
| Modified starch 1 - MCT | 97.42 | −0.09 | 1.45 | 266.4 |
| microcapsules (Batch 1) | | | | |
| Modified starch 1 - MCT | 97.43 | −0.09 | 1.95 | 278.2 |
| microcapsules (Batch 2) | | | | |
| Modified starch 2 - MCT | 97.48 | −0.11 | 1.43 | 305.0 |
| microcapsules | | | | |

### Example 2

Natural Gum Arabic-MCT Microcapsules at 40% MCT Loading Level

1. Heat 250 g of water to about 80° C.

2. Add 83.5 g of Gum Arabic to the hot water and agitate until Gum Arabic is dissolved.

3. Add 55.5 g of MCT oil to the solution.

4. Emulsify the solution for 5 minutes at 15,000 RPM to obtain an emulsion droplet size below 3 microns.

5. Pass the emulsified slurry through the microfluidizer at 15,000 psi to obtain an emulsion droplet size around one micron.

6. Spray dry the prepared emulsion with the following parameters:

a. Inlet Temperature: 160° C.

b. Blow Speed: 42 Hz

c. Feed Rate: 4 mL/min

d. Atomization: 0.25 mPa

e. Resulting Outlet Temperature should be 95-100° C.

### Example 3

Synthesis of Modified Starch-MCT at 40% Wt. Loading Level Microcapsules, Color Characterization, and Use in Whitening 2% Wt. Pectin Model System for Vegan Capsules

1. 360 g of modified starch was dissolved in 900 g of distilled water with stirring.

2. 240 g of MCT was added to the solution.

3. The solution was emulsified at 15,000 RPM, then passed through a microfluidizer to produce a fine, uniform emulsion of about 1 μm in size.

4. The emulsion was spray dried.

5. The resulting powder was packed with a vacuum sealer.

Similar modified starch-MCT microcapsules comprising different percentages of MCT were prepared by varying the amount of MCT added appropriately.

The microcapsules were characterized by measuring their color using the CIELAB model. The results are shown in Table 2.

TABLE 2

| Sample ID | L* | a* | b* |
|---|---|---|---|
| Example 1 | 97.96 | −0.19 | 1.57 |
| (20% MCT) | | | |
| 30% MCT | 97.33 | −0.09 | 1.74 |
| Example 3 | 97.90 | −0.14 | 1.43 |
| (40% MCT) | | | |
| Avalanche XTRA | 95.41 | −0.88 | −0.41 |
| NBS (Sensient) | | | |

The microcapsules were also used to whiten a 2% pectin suspension (a model for vegan capsules). The results are shown in Table 3.

TABLE 3

| Sample ID | L* | a* | b* |
|---|---|---|---|
| Control | 52.62 | 2.52 | 31.09 |
| Example 1 | 75.14 | 2.03 | 19.17 |
| (20% MCT) | | | |
| 30% MCT | 79.06 | 1.93 | 17.84 |
| Example 3 | 82.72 | 1.28 | 14.73 |
| (40% MCT) | | | |
| Avalanche XTRA | 77.83 | −0.33 | 18.11 |
| NBS (Sensient) | | | |

### Example 4

Another Batch of Natural Gum Arabic-MCT Microcapsules at 40% MCT Loading Level, Color Characterization, and Use in Lime Crema Application (a Salad Dressing)

300 g of gum arabic and 900 g of water were heated at 85° C. to dissolve the gum arabic. 200 g of MCT was added and the mixture was homogenized at 15,000 RPM for 8 min, then spray dried to produce a powder having L*=95.65, a*=0.07, and b*=3.99. The amount of MCT could be varied by changing the amount of MCT used in the preparation appropriately.

The gum arabic-MCT microcapsules were used to whiten lime crema. The results are shown in Table 4.

TABLE 4

| Sample ID | L* | a* | b* |
|---|---|---|---|
| Control | 76.51 | 1.04 | 23.02 |
| 30% MCT | 73.18 | 1.15 | 22.82 |
| 40% MCT VI | 74.28 | 1.53 | 22.99 |
| 40% MCT natural | 75.11 | 0.95 | 20.62 |
| Natural VI | 76.11 | 1.08 | 23.23 |
| V4 | 80.92 | 0.43 | 17.18 |
| VI | 75.14 | 1.03 | 23.98 |

Having now fully described the methods, microcapsules, and compositions herein, it will be understood by those of skill in the art that the same can be performed within a wide and equivalent range of conditions, formulations, and other parameters without affecting the scope of the methods, compounds, and compositions provided herein or any embodiment thereof. All patents, patent applications, and publications cited herein are fully incorporated by reference herein in their entirety.

1. A dry powder for whitening a food, or pharmaceutical product comprising spray-dried particles, wherein:
(i) the spray-dried particles comprise microcapsules; and
(ii) the microcapsules comprise an inner core and a primary shell surrounding the inner core, wherein (a) the inner core comprises food grade medium chain triglyceride oil; and (b) the primary shell comprises food grade modified starch or gum Arabic.

2. The dry powder of claim 1, wherein the redispersion diameter of the spray-dried particles is about 200 nm to about 5000 nm.

3. The dry powder of claim 1 having an L* value of at least 95 in the CIELAB color space.

4. The dry powder of claim 1 having an a* less than 0.15 in the CIELAB color space.

5. The dry powder of claim 1 having a b* value less than 5.0 in the CIELAB color space.

6. The dry powder of claim 1 that is stable.

7. A food comprising a sufficient amount of the dry powder of claim 1 to provide whitening to the food.

8. A pharmaceutical product comprising a sufficient amount of the dry powder of claim 1 to provide whitening to the pharmaceutical product.

9. The pharmaceutical product of claim 8 that is a chewable tablet.

10. The dry powder of claim 1 enhancing creaminess, richness and mouthfeel of food, beverage and pharmaceutical products.

11. The dry powder of claim 1 supporting weight loss and Alzheimer treatment due to a high level of MCT.
